(19) United States
(12) Patent Application Publication
(10) Pub. No.: US 2026/0226113 A1
(43) Pub. Date: Aug. 6, 2026

(54) SYSTEMS AND METHODS FOR PRODUCING HEXANE-FREE FOOD PRODUCTS AND INGREDIENTS THEREFOR

(71) Applicant: New Protein Canada Inc., Vaughan (CA)

(72) Inventor: Roi WURGAFT, Vaughan (CA)

(21) Appl. No.: 19/152,404

(22) PCT Filed: Mar. 1, 2024
(86) PCT No.: PCT/CA2024/050259

§ 371 Date: Jul. 30, 2025

Related U.S. Application Data
(60) Provisional application No. 63/488,126, filed on Mar. 2, 2023.

(51) Int. Cl. C07K 14/415; C07K 1/14

(52) U.S. Cl. CPC C07K 14/415; C07K 1/145

(57) ABSTRACT

Processes for preparing seed material such as for example soy beans into protein isolates and protein concentrates are provided. An example process for preparing a soy protein isolate comprises: providing defatted hexane-free soy flour pressed from soy material such as soybeans; extracting water from the hexane-free soy flour to produce an extraction slurry; adjusting the extraction slurry to have a pH in the range of 8.0 to 8.5 by adding sodium hydroxide to the extraction slurry and holding to produce pH-adjusted slurry; applying centrifugation to the pH-adjusted slurry to separate soy flour extracts from insoluble wet soy fiber material in the pH-adjusted slurry; adding a hydrochloric acid solution to the soy flour extracts to produce a low-pH mixture having a pH of about 4.5; mildly agitating the low-pH mixture; separating the low-pH mixture into a curd and a whey; diluting the curd with water and mixing with sodium hydroxide to produce a 10% dry solids mixture having a pH of about 7.0; pasteurizing the 10% dry solids mixture using a direct steam injection heater to produce a heated slurry; and cooling the heated slurry with a flash cooling system using a vacuumed tank to produce the soy protein isolate. The process may further comprise diluting the curd with water and mildly agitating to achieve a 6% dry solids mixture; and separating the 6% dry solids mixture into a second curd and a second whey, the second curd becoming the curd used in the diluting step.

### CROSS-REFERENCE TO RELATED APPLICATION

This patent application claims priority to U.S. provisional patent application No. 63/488,126 filed Mar. 2, 2023, the entire content of which is hereby incorporated by reference herein.

### TECHNICAL FIELD

The present invention relates to processing of plant materials, such as soybeans or other seeds, and in particular to food processing for food product production.

### BACKGROUND

Soybeans are an important food crop used in a wide variety of food products. Consumer demand for hexane-free high protein soy-based products having superior nutritional, functional, or organoleptic properties is on the rise. Soybean polar fats are typically being removed from high protein soy materials using traditional hexane oil extraction technology. These polar fats have nutritional benefits, which may include the presence of antioxidants and the ability to reduce cholesterol levels which supports the fight against heart disease, colon cancer, stomach cancer, obesity and heart attack. Polar fats may also serve as a source of choline, boost immunity, improve cognitive function, prevent osteoporosis, and help the body to deal with mental and physical stress.

In addition, the existence of polar fat has been found to improve emulsification, dispersibility, solubility, mouthfeel, and many others functional properties of soy protein. These added functional properties benefit food applications including meat and meat-analogs, soy-based beverages, sport nutrition, and health foods.

Due to the many benefits of polar fats, some existing hexane extracted soy protein products currently in the marketplace mix the polar fat material back into their high protein materials to achieve improved functionality and nutritional benefits.

Existing attempts to produce hexane-free high polar fat soy protein materials require the use of high temperature during the oil extraction process, typically done by conventional pressing, in order to remove residual non-polar oil that is not desired in the protein material. The non-polar oil must be removed as its presence leads to oxidation, off flavours, limited shelf life, and also interferes with and reduces the functionality of the protein material. Moreover, due to exposure to high temperature these conventional hexane-free oil extracted raw materials suffer from dark colour and protein denaturation. These characteristics have a negative effect both on the protein material quality and the ability to further concentrate or isolate the material due to a substantial reduction in yield.

There exists a need in the extraction process for a method to produce high quality hexane-free soy materials and soy-based food products without the reduction in protein caused by high temperature conventional hexane free oil pressing.

### SUMMARY

Processes for preparing soy material such as into soy protein isolates and soy protein concentrates are provided. In accordance with an aspect of the present disclosure, there is provided a process for preparing a soy protein isolate comprises: providing defatted hexane-free soy flour pressed from soy material such as soybeans; extracting water from the hexane-free soy flour to produce an extraction slurry; adjusting the extraction slurry to have a pH in the range of 8.0 to 8.5 by adding sodium hydroxide to the extraction slurry and holding to produce pH-adjusted slurry; applying centrifugation to the pH-adjusted slurry to separate soy flour extracts from insoluble wet soy fiber material in the pH-adjusted slurry; adding a hydrochloric acid solution to the soy flour extracts to produce a low-pH mixture having a pH of about 4.5; mildly agitating the low-pH mixture; separating the low-pH mixture into a curd and a whey; diluting the curd with water and mixing with sodium hydroxide to produce a 10% dry solids mixture having a pH of about 7.0; pasteurizing the 10% dry solids mixture using a direct steam injection heater to produce a heated slurry; and cooling the heated slurry with a flash cooling system using a vacuumed tank to produce the soy protein isolate.

In accordance with another aspect of the present disclosure, the process may further comprise diluting the curd with water and mildly agitating to achieve a 6% dry solids mixture; and separating the 6% dry solids mixture into a second curd and a second whey, the second curd becoming the curd used in the diluting step.

In accordance with another aspect of the present disclosure, there is provided a process for preparing a soy protein concentrate, the process comprising: providing defatted hexane-free soy flour pressed from soy material; mixing the hexane-free soy flour with water and adding a hydrochloric acid solution to produce a low-pH mixture having a pH of about 4.5; mildly agitating the low-pH mixture; separating the low-pH mixture into a curd and a whey; diluting the curd with water and mixing with sodium hydroxide to produce a 10% dry solids mixture having a pH of about 7.0; pasteurizing the 10% dry solids mixture using a direct steam injection heater to produce a heated slurry; and cooling the heated slurry with a flash cooling system using a vacuumed tank to produce the soy protein concentrate.

In accordance with another aspect of the present disclosure, there is provided a process for preparing a soy protein concentrate, the processing comprising: providing defatted hexane-free soy flour pressed from soy material; mixing the hexane-free soy flour with an aqueous ethanol solution in a 1:5 ratio and washed to produce an aqueous ethanol mixture; evaporating the aqueous ethanol mixture to recover soy sugar molasses, ethanol, and a protein fraction; and desolventizing the protein fraction to produce a soy protein concentrate.

In this respect, before explaining at least one embodiment of the present disclosure in detail, it is to be understood that the present disclosure is not limited in its application to the details of construction and to the arrangements of the components set forth in the following description or illustrated in the drawings. The inventive aspects of the present disclosure are capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

Non-limiting embodiments of the present disclosure will now be described, by way of example only, with reference to the attached figures, wherein:

FIG. 1 illustrates a flow chart describing a method of processing hexane-free soy-based materials in accordance with an aspect of the present disclosure;

FIG. 2 illustrates a flow chart describing a method of processing hexane-free soy-based materials in accordance with an aspect of the present disclosure;

FIG. 3 illustrates a flow chart describing a method of processing hexane-free soy-based materials in accordance with an aspect of the present disclosure; and

FIG. 4 illustrates a flow chart describing a method of processing hexane-free soy-based materials in accordance with an aspect of the present disclosure.

### DETAILED DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Some aspects of present disclosure provide methods of processing hexane free soy-based materials suitable for use in a wide variety of applications. In an example process, soy material is extracted by a low temperature press, such as, for example, a type of press described in U.S. Pat. No. 7,687,648 to Smallridge et al. After extraction, the soy material is milled, texturized, concentrated, and optionally passed through an isolation extraction process (e.g. water extraction, aqueous alcohol extraction, ultra-filtration, aqueous fat separation or any other extraction process) to produce an enriched protein fraction and in some processes an additional enriched fiber fraction. In aspects of the present invention, the enriched protein fraction may contain a minimum of 50% protein content on a dry basis (measured by Kjeldahl method AOAC 18th Ed. Method 991.2.2, which is incorporated herein by reference in its entirety) and the enriched fiber fraction may contain a maximum 40% dry basis Kjeldahl protein content. The produced protein material and fiber material are then modified to achieve the required functional properties based on the final application by means of pH adjustment, heat treatment, homogenization, de-flavoring, bleaching, enzymatic treatment, or any other known techniques for functional modification of protein and fiber materials. The protein or fiber material is then blended into a final hexane free soy-based product formulation to produce a composition with improved functionality, color and flavor.

The protein material may be further concentrated by separating the soluble sugars fraction to a minimum Kjeldahl protein content of 65% (on dry basis), such as, for example, by acid precipitation, ultra-filtration, salt-out or any other well-known protein concentration methods, and thereafter may undergo the same functional modification stage as describe above.

Processes according to some aspects of the present disclosure result in improved functionality for whole muscle injected meat product prepared from the protein material provided. The improved whole muscle injected meat product may have better water retention, flavor, dispersibility and other functional properties compared to the same whole muscle injected meat product produced from conventional soy protein material.

Processes according to some aspects of the present disclosure also provide improved functionality for high protein beverage products. The improved beverage product has better flavor, color, protein solubility, and other functional properties when compared to conventional soy beverage products.

An improved functional high protein non-dairy yogurt product can be prepared from the protein material that is produced according to processes of the present disclosure. The improved non-dairy yogurt product has better nutrition flavor, color, protein solubility, mouthfeel, and other functional properties when compared to conventional soy non-dairy yogurt products.

An improved functional high protein soy crisp product can be prepared from the protein material that is produced according to processes of the present disclosure. The improved soy crisps product has better nutrition, flavor, color, texture and other functional properties when compared to conventional soy crisp product.

An improved functional high protein nutritional bar soy crisp product can be prepared from the protein material that is produced according to processes of the present disclosure. The improved nutritional product has better nutrition, flavor, color, texture, and other functional properties when compared to conventional soy nutrition bar product.

An improved textured soy protein concentrate product can be prepared from the protein material that is produced according to processes of the present disclosure. The improved textured soy protein concentrate product may have better nutrition, flavor, color, texture and other functional properties when compared to conventional textured soy protein concentrate product.

Types of soy-based materials that may be prepared according to the present disclosure include, without limitation: (1) soy protein material containing at least 50% dry basis protein; (2) soy protein material containing at least 65% dry basis protein; (3) soy protein material containing at least 85% dry basis protein; and (4) soy fiber composition containing maximum 35% dry basis protein.

The soy based materials prepared by processes according to the present disclosure can be used to make a wide variety of soy based food products. These food products include, but are not limited to, confectionary products, bakery products, injection meat products, emulsified meat products, ground meat products, meat analog products, cereals, cereal bars, dairy analog products, beverages, soy milk, dietetic formula, pasta, health nutrition supplements, and nutrition bars. In particular, a confectionary product may include, but is not limited to, candy or chocolate. A bakery product may include, but is not limited to, breads, rolls, biscuits, cakes, yeast baked goods, cookies, pastries, or snack cakes. An injection meat product includes, but is not limited to ham, poultry products, turkey product, chicken product, seafood product, pork product or beef product. An emulsified meat product includes, but is not limited to sausage, bratwurst, salami, bologna, lunchmeat, or hot dogs. A ground meat product includes, but is not limited to fish sticks, meat patties, meatballs, ground pork products, ground poultry products, ground seafood products or ground beef products. A meat analog product includes, but is not limited to sausages, patties, ground meatless crumbles, lunchmeat or hot dogs. A dairy analog product includes, but is not limited to milk products, yogurt products, sour cream products, whipped topping, ice cream, cheese, shakes, coffee whitener or cream products. A dietetic formula includes, but is not limited to infant formula, geriatric formula, weight loss preparations, weight gain preparations, sports drinks, or diabetes management preparations.

An almost infinite number of the food products may be made by altering the improved functionality of the soy materials in the food product. Persons skilled in the art may modify the type and content of proteins, sugar sources, fats and oils, vitamin/mineral blends, flavors, gums, and/or flavors to produce a beverage product designed to meet specific nutritional requirements, product marketing claims, or targeted demographic groups. For example, nutritional bars may be produced using the improved functionality soy material as a partial or complete protein source. Persons skilled in the art may modify the type, texture, and content of proteins, sugar sources, fats and oils, vitamin/mineral blends, flavors, coatings gums, and/or flavors to produce a nutritional bar designed to provide specific compositions to meet specific nutritional requirements, product marketing claims, or targeted demographic groups.

Any suitable soy raw material may be used in processes according to the present disclosure. Suitable soy raw materials include, but are not necessarily limited to: whole soybeans, and full-fat soy material (for example, a soy material that has not been defatted prior to milling). The term “soy material” as used herein is intended to encompass at least these soy raw materials. In some embodiments, processes according to the present disclosure may use other types of seeds or other plant-based protein material, including without limitation canola seeds, hemp seeds, and mustard seeds. The term “seed material” as used herein is used to encompass at least these types of plant-based protein materials.

The examples described herein represent only part of the wide variety of the improved functionality soy materials and the improved functionality soy food products. The improved functionality soy materials have some functional properties considered to be desirable in soy based food products. The following functional properties are currently being evaluated for the improved functionality wet soy materials: water binding ability, fat binding, emulsification, gel strength, solution particle size, solubility, dispersibility, viscosity, color and taste as well as others.

Protein:water gel strength is a measure of the strength of a refrigerated gel made using a soy protein composition. The strength of the gel is measured with a TX-TI texture analyzer which drives a cylindrical probe into the gel until the gel is ruptured by the probe and calculating the gel strength in grams from the recorded break point of the gel. Amongst the many applications for high gel food ingredients are meat emulsions, meat analogs, yogurt, imitation cheese, and other products where the ability to form a protein gel in water is desired.

Protein:oil:water emulsion strength is a measure of the strength of a refrigerated oil and water emulsion with soy protein. The strength of the emulsion is measured with a TX-TI texture analyzer which drives a cylindrical probe into the emulsion until the emulsion is ruptured by the probe and calculating the emulsion strength from the recorded break point of the emulsion. High emulsion strength is required to produce meat emulsion products and other food products that require emulsification properties.

Fat Emulsion separation is a quality measure of a cooked high fat meat formulation including the soy material. Thereafter cooking the high fat meat product a quality determination of fat separation is done in order to determine the ability of the soy material to hold fat in meat formulation.

RVA viscosity method is used for products that dedicated to be use in: whole muscle meat injection, RTD beverages, and any other applications that required viscosity control. The RVA viscosity method uses a slurry with specific dry solids percentage to calculate its viscosity profile using a Rapid Visco Analyzer (RVA). The RVA is used to measure the apparent viscosity of the slurry. Viscosity profile for soy based food product is required for meat injection RTD beverages and other applications.

The Nitrogen Solubility Index (NSI) determines the relative solubility of the soy protein materials. A low NSI, on a scale of 0-100%, indicates low protein solubility and a high NSI indicates high protein solubility. The NSI method is the official methods and recommended practices of the AOCS, 5th Edition, Method Bc 4-49. NSI value is required for applications such as: RTD beverages, whole muscle meat injection, soy milk and others.

Sedimentation rate is a method that used for products that dedicated to be use in: meat injection, RTD beverages and others. This method analyzes the percentage of protein sedimentation after specific time at specific conditions.

Other methods that may be used in order to define the quality of the improved functionality soy materials are: colour (l, a, b); particle size profile in wet slurry; odour and flavour profile.

Various exemplary processes in accordance with aspects of the present disclosure will now be described with reference to the figures. These examples are meant only to be illustrative and are not intended to be limiting.

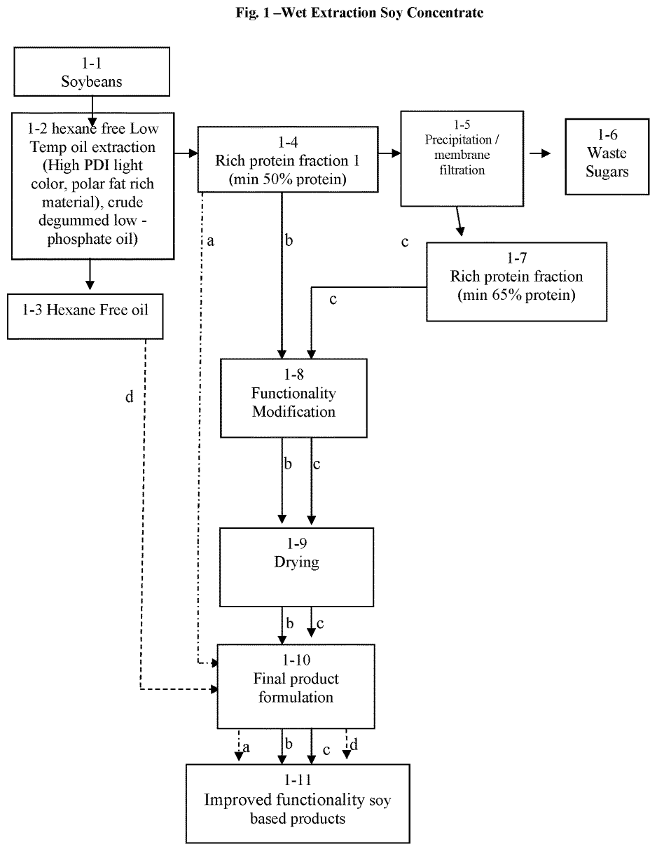

FIGS. 1-4 are flowcharts illustrating methods for producing hexane-free food products according to example embodiments of the present disclosure. FIGS. 1-4 show various streams of products processed according to methods of the present disclosure, as summarized in the following table:

| FIG. | Stream | Description |
|---|---|---|
| 1 | a | Defatted Flour after CO₂ press - sold as is to the market |
| | b | Functional Defatted soy flour after rehydration, functionalization and spray |
| | | drying |
| | c | Functional fractionated soy protein concentrate produced from the defatted |
| | | material thru water fractionation (removal of sugars) |
| | d | Crude Degummed Low Phosphate hexane free oil from the CO2 press |
| 2 | a | Functional fractionated soy protein isolate produced from the defatted |
| | | material thru water fractionation (removal of fibers and/or sugars) |
| | b | Functional Soy Fibers after drying |
| | c | non-functional soy fibers in a wet or dry form |
| | d | Soy Molasses sugars syrup rich with phospholipids |
| 3 | a | Soy Molasses sugars syrup rich with phospholipids |
| | b | Aqueous Ethanol extracted Soy Protein Concentrate (SPC) |
| | c | Texturized Soy Protein Concentrate produced from Steam b SPC |
| | d | Functional Soy Protein Concentrate produced from Steam b SPC |
| 4 | a | Textured Soy Protein produced from Defatted Flour after CO₂ press |

In FIG. 1, the method starts with soybeans, which are dehulled at step 1-1 then fed to a press to remove oil at step 1-2 to produce a hexane free oil (step 1-3) and a first rich protein fraction having at least 50% protein (step 1-4). The first rich protein fraction can be subjected to precipitation/membrane filtration (step 1-5) to produce waste sugars (1-6) and a second rich protein fraction having at least 65% protein (step 1-7), and or proceed directly to functionality modification at step 1-8. The second rich protein fraction is also subjected to functionality modification at step 1-8. At step 1-9 the produce is dried before being subjected to final product formulation at step 1-10 to produce improved functionality soy based products at step 1-11.

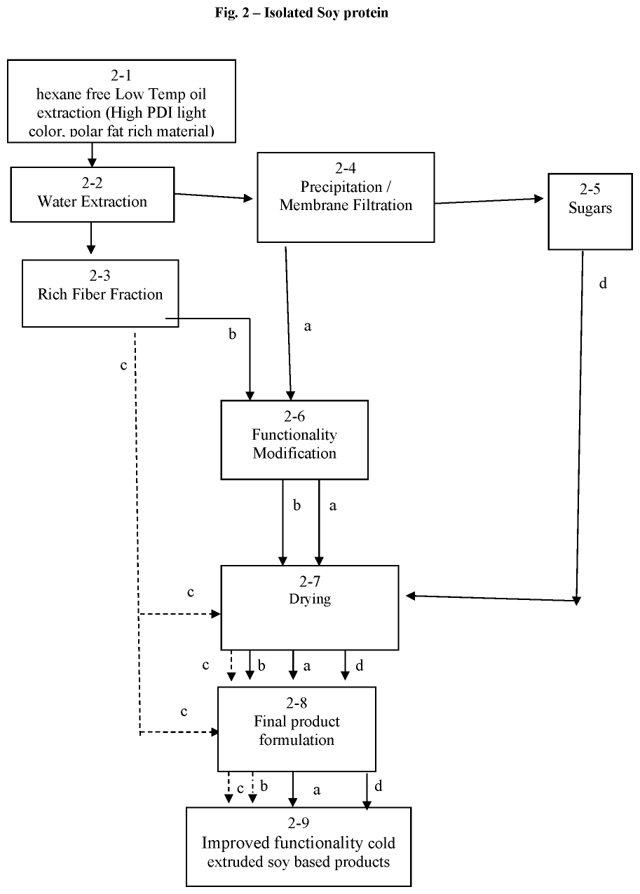

In FIG. 2, the method stars with dehulled pressed soybeans at step 2-1, water is extracted at step 2-2 and the material is separated to produce a rich fiber fraction (step 2-3) and soy flour extracts which are subjected to precipitation/membrane filtration at step 2-4 to produce a soy protein isolate, along with sugars (step 2-5). The soy protein isolate and rich fiber fraction are subjected to functionality modification at step 2-6, and drying at step 2-7. Other streams are also optionally dried at step 2-7 and all product streams are then subjected to final product formulation at step 2-8 to produce improved functionality soy based products at step 2-9.

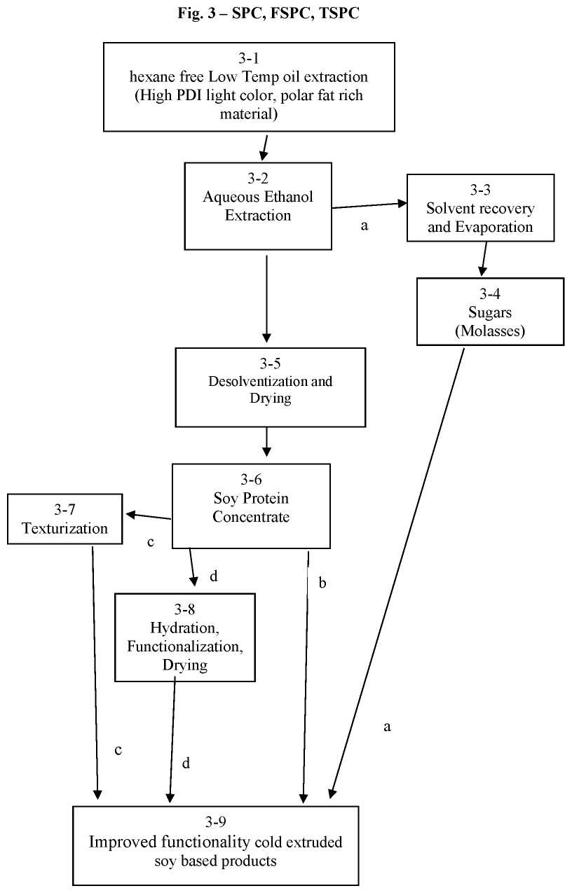

In FIG. 3, the method stars with dehulled pressed soybeans at step 3-1, and the pressed material is subjected to aqueous ethanol extraction at step 3-2 and solvent recovery at step 3-3 to extract a soy molasses sugar syrup rich with phospholipids. The remaining protein fraction is desolventized and dried at step 3-5 to produce a soy protein concentrate at step 3-6. The soy protein concentrate can optionally be subjected to texturization at step 3-7, subjected to hydration, functionalization and drying at step 3-8, and be provided directly as an improved functionality soy based products at step 3-9.

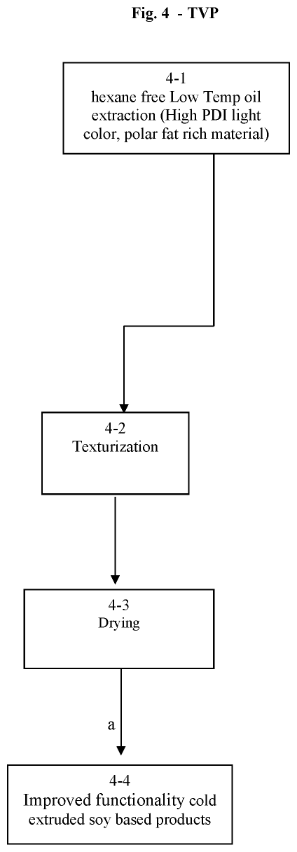

In FIG. 4, the method stars with dehulled pressed soybeans at step 3-1, and the pressed material is subjected to texturization at step 4-2. After texturization, the material is dried at step 4-3, to produce a textured soy protein with improved functionality at step 4-4.

Detailed example methods of preparation of food products, and formulations thereof, are set forth below.

### Example 1: Preparation of Hexane-Free Soy Protein Isolate (“SPI”)

In this example, a soy protein isolate is prepared according to a process of the present disclosure. The process begins by providing defatted hexane-free soy flour pressed from soy material, such as soybeans. The press used may be a screw press configured for injection of a solvent. The solvent injected into the press comprises carbon dioxide in some embodiments. In other embodiments, different types of hexane-free solvents may be injected into the press, such as for example an inert non-carbon gas such as nitrogen. With reference to FIGS. 1 and 2, when starting from soybeans, the soybean hulls may be removed, and the dehulled soybeans fed to the press to remove oil.

Water is extracted from the hexane-free soy flour to produce an extraction slurry. The hexane-free soy flour may have a total of 16 parts water at 55 degrees Celsius to each part of soy flour. The extraction slurry is adjusted to have a pH in the range of 8.0 to 8.5 by adding sodium hydroxide to the extraction slurry and holding, for example a mean time of 30 minutes, to produce pH-adjusted slurry.

The soy flour extracts are then separated from the insoluble wet soy fiber material. This may be done by using a high g-force centrifugation to the pH-adjusted slurry. The extract is then precipitated by adding a hydrochloric acid solution, such as a 20% hydrochloric acid solution, to the soy flour extracts to produce a low-pH mixture having a pH of about 4.5 at 50 degree Celsius. The low-pH mixture may be held for approximately 30 minutes with mild agitation. Then the low-pH mixture may be separated into a curd (precipitated protein) and a whey (sugars), such as by centrifugation. In some embodiments, the separation is performed utilizing a continuous discharge centrifuge.

Optionally, the curd may be diluted with water and mildly agitated to achieve a 6% dry solids mixture (measured by AOAC method 927.05) at 50 degree Celsius. The 6% dry solids mixture may then be held for about ten minutes with mild agitation, and then centrifuged to separate into a second curd (precipitated protein) and second whey (sugars).

Either the original curd, or this optional second curd may then be diluted with water and mixing with sodium hydroxide to produce a 10% dry solids mixture having a pH of about 7.0. The 10% dry solids mixture may be processed through a pasteurization stage in a continuous process using a direct steam injection heater, for example, heated up to 135 degree Celsius, to produce a heated slurry. The heated slurry may be hold for about 3 seconds and thereafter cooled down, for example to about 55 degrees Celsius, by a flash cooling system using a vacuumed tank to produce a soy protein isolate. The soy protein isolate may then be immediately fed to a spray drier to produce a soy protein isolate powder product having moisture content of about 5.5%.

The process yields, composition and functionality of a soy protein isolate produced according to Example 1 were analyzed. The results showed that by using low temp high protein dispersibility index (“PDI”) hexane-free defatted flour, a soy protein isolate having at least 85% protein and less than 10% acid hydrolysis fat, of which under 2% is free fat and the rest is a combination of polar fats (sterols, phospholipids and other gums) was obtained.

### Example 2: Preparation of Hexane-Free Soy Protein Concentrate (“SPC”)

In this example, a soy protein concentrate is prepared according to a process of the present disclosure. The process begins by providing defatted hexane-free soy flour pressed from soy material, such as soybeans. The soy flour may be low temperature and high PDI. The press used may be a screw press configured for injection of a solvent. The solvent injected into the press comprises carbon dioxide in some embodiments. In other embodiments, different types of hexane-free solvents may be injected into the press, such as for example an inert non-carbon gas such as nitrogen. With reference to FIGS. 1 and 2, when starting from soybeans, the soybean hulls may be removed, and the dehulled soybeans fed to the press to remove oil.

The hexane-free soy flour is mixed with water and then precipitated by adding 20% hydrochloric acid solution to produce a low-pH mixture having a pH of about 4.5 at 50 degree Celsius.

The low-pH mixture may be held for approximately 30 minutes with mild agitation. Then the low-pH mixture may be separated into a curd (precipitated protein) and a whey (sugars), such as by centrifugation.

Optionally, the curd may be diluted with water and mildly agitated to achieve a 6% dry solids mixture (measured by AOAC method 927.05) at 50 degree Celsius. The 6% dry solids mixture may then be held for about ten minutes with mild agitation, and then centrifuged to separate into a second curd (precipitated protein) and second whey (sugars). In some embodiments, the separation is performed utilizing a continuous discharge centrifuge.

Either the original curd, or this optional second curd may then be diluted with water and mixing with sodium hydroxide to produce a 10% dry solids mixture having a pH of about 7.0. The 10% dry solids mixture may be processed through a pasteurization stage in a continuous process using a direct steam injection heater, for example, heated up to 135 degree Celsius, to produce a heated slurry. The heated slurry may be hold for about 3 seconds and thereafter cooled down, for example to about 55 degrees Celsius, by a flash cooling system using a vacuumed tank to produce a soy protein concentrate.

The soy protein concentrate may then be immediately fed to a spray drier to produce a soy protein concentrate powder (awFSPC) product having moisture content of about 5.5%.

The process yields, composition and functionality of an soy protein concentrate produced according to Example 2 were analyzed. The results showing that by using low temp high PDI hexane-free defatted flour, a soy protein concentrate having at least 65% protein and less than 10% acid hydrolysis fat, of which under 2% is free fat and the rest is a combination of polar fats (sterols, phospholipids and other gums) was obtained.

### Example 3: Preparation of Hexane-Free Aqueous Ethanol Washed SPC

In this example, and with reference to FIG. 3, a hexane-free aqueous ethanol washed soy protein concentrate is prepared according to a process of the present disclosure. The process begins by providing defatted hexane-free soy flour pressed from soy material, such as soybeans. The soy flour may be low temperature and high PDI. The press used may be a screw press configured for injection of a solvent. The solvent injected into the press comprises carbon dioxide in some embodiments. In other embodiments, different types of hexane-free solvents may be injected into the press, such as for example an inert non-carbon gas such as nitrogen. When starting from soybeans, the soybean hulls may be removed, and the dehulled soybeans fed to the press to remove oil.

The hexane-free soy flour is mixed with an aqueous ethanol solution, for example a 68% aqueous ethanol solution, in a 1:5 ratio and washed, for example, 6 times, to produce an aqueous ethanol mixture.

The miscella/aqueous ethanol mixture is evaporated to recover soy sugar molasses, ethanol, and a protein fraction. The ethanol may be recycled.

The protein fraction is desolventized, for example, under low temperature (under about 65 degrees Celsius) to produce a traditional soy protein concentrate. The traditional soy protein concentrate was measured to have >67% protein and <8% acid hydrolysis fat, of which <1% is free fat and the rest is a combination of polar fats (sterols, phospholipids and other gums).

### Example 4: Preparation of Hexane-Free Functional SPC

In this example, and with reference to FIG. 3, a hexane-free functional soy protein concentrate (“FSPC”) is prepared according to a process of the present disclosure. The soy protein concentrate from Example 3 is used as a raw material to produce the FSPC in this example. The soy protein concentrate is mixed with water and sodium hydroxide is added to produce a soy protein mixture with a pH of 7.0. The mixture is hydrated to 10% solids with water at 55 degrees Celsius.

The soy protein mixture is pasteurized using a direct steam injection heater (for example, heated up to 135 degree Celsius). The heated slurry may be held for about 3 seconds and thereafter cooled down by flash cooling system using a vacuumed tank (for example, down to about 55 degree Celsius) to produce the functional soy protein concentrate. The functional soy protein concentrate may then be immediately fed to a spray drier to produce a FSPC powder product having moisture of 5.5%.

The composition and functionality of a FSPC produced according to Example 4 were analyzed. The results showing that by using SPC produced from low temp high PDI hexane free defatted flour, a functional soy protein concentrate having at least 65% protein and less than 8% acid hydrolysis fat, of which under 1% is free fat and the rest is a combination of polar fats (sterols, phospholipids and other gums) was obtained.

### Example 5: Preparation of Hexane-Free Textured SPC

In this example, and with reference to FIG. 3, a hexane-free textured soy protein concentrate (“TSPC”) is prepared according to a process of the present disclosure. The soy protein concentrate from Example 3 is used as an input material to produce the TSPC in this example. The soy protein concentrate is mixed with water and direct steam in a pre-conditioner, to produce an 80% solids mixture. The 80% solids mixture is fed into a twin screw extruder to produce extruded material. The twin screw extruder may be of a type, for example, such as by Wenger. The extruded material may then be cut into textured pieces, such as into textured chunks and flakes. A shredder machine may be used for the cutting, for example, such as a Fitz mill type shredder machine. The textured pieces are then dried in a fluid bed air dryer to produce a textured soy protein concentrate.

The product, composition and functionality of a TSPC produced according to Example 5 were analyzed. The results showed that by using SPC produced from low temp high PDI hexane free defatted flour according to Example 3 as the input material, a soy textured soy protein concentrate having at least 65% protein and less than 8% acid hydrolysis fat, of which under 1% is free fat and the rest is a combination of polar fats (sterols, phospholipids and other gums) was obtained.

### Example 6: Preparation of Hexane-Free Textured Soy Protein

A textured soy protein (“TSP”) is prepared according to a process of the present disclosure. The process begins by providing defatted hexane-free soy flour pressed from soy material, such as soybeans. The soy flour may be low temperature and high PDI. The press used may be a screw press configured for injection of a solvent. The solvent injected into the press comprises carbon dioxide in some embodiments. In other embodiments, different types of hexane-free solvents may be injected into the press, such as for example an inert non-carbon gas such as nitrogen. With reference to FIG. 4, when starting from soybeans, the soybean hulls may be removed, and the dehulled soybeans fed to the press to remove oil.

The soy flour is used as a raw material to produce textured soy protein by hydration to 80% solids with water and direct steam in a pre-conditioner follow by feeding the material into a twin screw extruder (Wenger). The extruded material is cut into textured chunks and flakes by a Fitz mill type shredder and then dried in a fluid bed air dryer to produce textured soy protein. The product, composition and functionality of the TSP were analyzed.

The results showing that by using the low-temperature high-PDI hexane-free defatted soy flour, a soy textured soy protein having >50% protein and <8% acid hydrolysis fat, of which <2% is free fat and the rest is a combination of polar fats (sterols, phospholipids and other gums) was obtained.

### Example 7: Preparation of Soy Drink Product

A soy drink produce is prepared from the soy protein isolate of Example 1 produced by a process of the present disclosure by blending with other ingredients. The soy drink product (soy milk) is prepared according to the a formulation as follows: 88.74% water; 2.95% soy protein isolate from example 1; 1.5% soybean oil; 4.5% sugars; 1.00% gums; 1.30% vitamin/mineral fortification; and 0.01% flavourings.

After the addition of the above ingredients, the soy drink formulation is pasteurized, chilled and bottled in glass containers. The final soy drink product prepared with the SPI material of Example 1 has bland flavor, low sedimentation and high protein solubility.

### Example 8: Preparation of Emulsified Sausages

Emulsified sausages are prepared using the FSPC produced in Examples 2 and 4. The FSPC is mixed in a food processor together with ground meat (about 30% fat), starch and phosphate to produce meat sausages paste. The meat sausage formulation is as follows: 2.7% FSPC product; 24.6% water; 67.9% meat (30% fat); 1.9% starch; and 2.9% phosphates. The paste is then stuffed in sausage casing and cooked at 85 degrees Celsius. The cooked sausage is chilled down overnight and then stripped and analyzed for fat separation, mouthfeel and flavor profile. It was observed that the meat sausage prepared using FSPC material has no fat separation; bland flavor and strong meat bite.

### Example 9: Preparation of Meat Brine

Whole muscle meat injection brine is prepared using SPI or FSPC materials of Example 1, 2 and 4. Meat brines (125% and 150%) may be prepared using the SPI produced in Example 1 or the FSPC material produced in examples 2 and 4 in order to increase juiciness and yield of a lean ham or whole muscle meat product by injection. The brines are prepared by adding extra water and other ingredients. A 125% in brine may have a composition as follows: 82% ice water; 9% salt; 3% STTP; 0% carrageenan; and 6% protein. A 150% in brine may have a composition of 88% ice water; 4.5% salt; 1.5% STTP; 1.5% carrageenan; and 4.5% protein. The resulting injected meat pieces using this brine will have a firm bite and dry surface with no visible strips or pockets of the injected brine.

### Example 10: Preparation of Meat Analog Patties

Meat analog patties are prepared using the SPI or FSPC materials of Example 1, 2, and 4 and the textured material from Examples 5 and 6. The composition of the meat analog patties is as follows: 36% water; 4% soy protein material; 7% wheat gluten; 15% soybean oil; 1.5% methyl cellulose; 0.3% cane sugar; 25% textured soy protein; 9.2% extra water; 0.4% sodium carbonate; 1.2% salt; 0.4% seasoning (Ogawa Beef Flavor #B18538, 2.8 Ogawa, Tokyo, Japan).

The Textured soy material is soaked with part of the water and kept refrigerated separately. Sodium carbonate and water is then mixed in a food cutter (Hobart Manufacturing Co., model 84145, Troy, Ohio) for two minutes. The soy protein materials are then added (each one in a separate formula) to the mixture and mixed one minute and the mixture is then refrigerated at 4-6° C. The extra water is heated to 80° C. and chopped on high speed with the methylcellulose for one minute in the same Hobart cutter. The soybean oil is added slowly with high speed chopping and chopped one minute. The remaining ingredients are added and chopped 3 minutes. The refrigerated textured-fiber-protein-sodium carbonate mixture is then added to the emulsion and mixed two minutes. The mixture is formed into patties using a Formax F-6 former (Formax Inc., Mokena, Ill.). Patties are flash frozen at −40° C.

### Example 11: Preparation of Soy-Based Yogurt Analog

A soy-based yogurt analog is prepared using the SPI or FSPC materials of examples 1, 2, and 4 according to the following ingredients: 34.5% sweet dairy whey; 3.35% soy material; 29.65% water; 26.2% vegetable oil; 3.0% sugar; 1.5% emulsifiers; 1.0% vitamins/minerals; and 0.5% flavouring. All oils for the tests are combined in a tank and heated to 70° C., and the emulsifiers are added. The SPI or FSPC materials are mixed with water (each one in a separate formula) in a separate tank and heated to 49° C. The whey and sugars are then added and blended for 15 minutes prior to the addition of the oil with emulsifiers. The solution is then heated to 90° C. for 5 minutes, homogenized in a two-stage homogenizer at 2500 and 500 psi respectively and then cooled to 35° C. After the entire mixture reaches 35° C., a 2% standard yogurt starter culture is inoculated. The temperature is maintained at 35° C. until the pH of the mixture reaches 4.6, then the vitamins, minerals, and flavorings are added, and the mixture is cooled to 4° C. for packaging.

### Example 12: Preparation of Frozen Desert

A frozen desert can be made using the SPI or FSPC materials of examples 1, 2, and 4. The ingredients are as follows: 10% hydrogenated soybean oil; 7% soy protein material; 63% water; 8% corn syrup solids 42 dextrose equivalent; 12% sucrose; and 0.75% stabilizer blend. The stabilizer blend may be composed as follows: 72.45% cellulose gum; 8.7% carboxymethyl cellulose; 7.25% locust bean gum; 5.8% xanthan gum; and 2.9% carrageenan. The soy protein materials and water are added (each one in a separate formula) to an agitated tank and heated to 54° C. All other dry ingredients are added to the slurry under sufficient agitation for complete mixing. The hydrogenated soybean oil is added under the same agitation until thoroughly mixed. The solution is pasteurized at 78° C. for 20 seconds and homogenized at 100/33 bar. The mixture is frozen with 70 to 100% overrun and is then packaged to harden.

### Example 13: Preparation of Infant Formula

A soy-based infant formula may be made using the SPI material of Example 1. The ingredients used in the formulation and the procedure are as follows: 1.5% SPI material; 13.5% water; 35% 42 dextrose equivalent corn syrup solids; 21% sucrose; 3% vitamins and minerals; 14% corn oil; 10.6% coconut oil; and 1.4% emulsifier. The vegetable oils are heated to 66° C. then the emulsifier is added. In a separate tank, water and SPI product is stirred at 49° C. with adequate agitation. Neutrase enzyme (or other suitable protease) is added at 0.1% of protein weight under constant agitation for one hour to hydrolyze the protein in the soy solution. The solution is pasteurized after an hour to stop the reaction by denaturing the enzyme. All other ingredients are added and blended for 15 minutes before the oil with emulsifier is added. After the entire mixture is blended for an additional 15 minutes, it is homogenized, pasteurized and spray dried. The soy based infant formula performs well in the ability to emulsify fat and remain soluble after rehydration.

### Example 14: Preparation of Beverage

A ready to drink beverage may be made using the SPI and FSPC materials of Example 1, 2, and 4 according to the following formulation: 8.8% soy protein composition; 79.2% water; 10% sucrose; 0.45% cocoa; 0.5% vitamins/minerals; 0.5% flavour; and 0.45% cellulose gel. Water and soy protein materials are mixed (each one in a separate formulation) at 60° C. under strong agitation. The cocoa is pre-blended with the cellulose gel and the sugar, then added to the protein water mixture and the final vitamins, minerals, and flavors are added. The mixture is homogenized, pasteurized, and packaged in aseptic or retort containers. One 240 ml serving of the high protein, ready to drink beverage will supply 20 grams of protein per serving.

### Example 15: Preparation of Food Bar

A food bar may be produced using the SPI material of Example 1 and the textured soy proteins from Examples 4 and 5 using the following composition: 15% powder SPI; 10% textured soy protein; 40% corn syrup; 10% rice syrup solids; 3% glycerin; 5% cocoa; and 17% compound coating. The textured soy material and the SPI are added together to corn syrup at 160° C. in a high shear mixer until fully blended. The glycerin and rice syrup are added until the mixture is fully mixed. The temperature is increased to 95° C. in a scraped surface heat exchanger and held for 5 minutes, then fed to an extruder/former to shape a continuous bar with width of 40 mm and a height of 20 mm. The continuous rectangular bar is cut into lengths of 100 mm in a continuous process creating a food bar with 60 grams weight. Separately, the cocoa is added to the coating compound and heated to 70° C. The coating compound mixture is pumped onto the moving continuous bar such that 10 grams of coating is applied to each 60-gram bar. The 70-gram bar is cooled to 25° C. by blowing conditioned air onto the moving product, and the food bar is packaged in a metallic coated flexible packaging as a hexane free protein rich nutritional bar containing 15 grams of protein per bar.

### Example 16: Preparation of Soy Protein Crisps

The SPI from Example 1 may be used as a raw material to produce soy protein crisps, by mixing with 20% native corn starch and feeding the material into a twin screw extruder (Wenger). The extruded material is cut into small round balls (2-3 mm) that are then used as high protein soy crisps in nutritional bar applications.

Although the above examples are directed to preparation of food product using soy materials, it is to be understood that techniques discloses herein can also be used to prepare food products using other plant-based materials. For example, in some embodiments a seed material comprising soft seeds such as canola seeds, hemp seeds, mustard seeds, or any combination thereof can be used as a starting material in place of, or in combination with, the soy material.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. (canceled)

2. (canceled)

3. (canceled)

4. (canceled)

5. (canceled)

6. The process of claim 21 wherein the 10% dry solids mixture is heated to about 135 degrees Celsius by the direct steam injection heater to produce the heated slurry.

7. The process of claim 21 wherein the heated slurry is cooled by the flash cooling system down to about 55 degrees Celsius.

8. The process of claim 21 wherein the seed material comprises a soy material, and the seed protein isolate is a soy protein isolate.

9. A process for preparing a seed protein concentrate, the process comprising:
pressing a seed material using a screw press and injecting a gas into the screw press to produce a defatted hexane-free seed flour;
mixing the hexane-free seed flour with water and adding a hydrochloric acid solution to produce a low-pH mixture having a pH of about 4.5;
mildly agitating the low-pH mixture;
separating the low-pH mixture into a curd and a whey;
diluting the curd with water and mixing with sodium hydroxide to produce a 10% dry solids mixture having a pH of about 7.0;
pasteurizing the 10% dry solids mixture using a direct steam injection heater to produce a heated slurry; and
cooling the heated slurry with a flash cooling system using a vacuumed tank to produce the seed protein concentrate.

10. The process of claim 9 wherein injecting the gas comprises injecting carbon dioxide into the screw press.

11. The process of claim 9 wherein injecting the gas comprises injecting a non-carbon gas into the screw press.

12. The process of claim 9 further comprising, prior to the diluting step:
diluting the curd with water and mildly agitating to achieve a 6% dry solids mixture; and
separating the 6% dry solids mixture into a second curd and a second whey, the second curd becoming the curd used in the diluting step.

13. The process of claim 9 further comprising spray drying the seed protein concentrate to produce a seed protein concentrate powder product having a moisture content of about 5.5%.

14. The process of claim 9 wherein the seed material comprises a soy material, and the seed protein concentrate is a soy protein concentrate.

15. A process for preparing a seed protein concentrate, the processing comprising:
pressing a seed material to obtain a defatted hexane-free seed flour;
mixing the hexane-free seed flour with an aqueous ethanol solution having a concentration of about 68% to produce an aqueous ethanol mixture;
washing out and evaporating the aqueous ethanol mixture to recover soy sugar molasses, an ethanol stream for recycling, and an insoluble protein concentrate fraction; and
desolventizing and drying the insoluble protein concentrate fraction under vacuum conditions at a low temperature to produce a seed protein concentrate.

16. The process of claim 15 wherein the desolventizing step is performed at a temperature less than about 65 degrees Celsius.

17. The process of claim 15 further comprising:
mixing the seed protein concentrate with water and adding sodium hydroxide to produce a seed protein mixture with a pH of 7.0;
pasteurizing the seed protein mixture using a direct steam injection heater to produce a heated slurry;
cooling the heated slurry with a flash cooling system using a vacuumed tank to produce a functional seed protein concentrate.

18. The process of claim 17 further comprising feeding the functional seed protein concentrate to a spray drier to produce a seed protein concentrate powder.

19. The process of claim 15 further comprising:
mixing the seed protein concentrate with water in a pre-conditioner, to produce an 80% solids mixture;
feeding the 80% solids mixture into a twin screw extruder to produce extruded material;
cutting the extruded material into textured pieces; and
drying the textured pieces in a fluid bed air dryer to produce a textured seed protein concentrate.

20. The process of claim 15 wherein the seed material comprises a soy material, and the seed protein concentrate is a soy protein concentrate.

21. A process for preparing a seed protein isolate powder product, the process comprising:
feeding a seed material into a screw press and injecting a gas into the screw press to produce a defatted hexane-free seed flour;
subjecting the hexane-free soy flour to water extraction to produce an extraction slurry;
adjusting the extraction slurry to have a pH in the range of 8.0 to 8.5 by adding sodium hydroxide to the extraction slurry and holding to produce pH-adjusted slurry;
applying centrifugation to the pH-adjusted slurry to separate seed flour extracts from insoluble wet seed fiber material in the pH-adjusted slurry;
adding a hydrochloric acid solution to the seed flour extracts to produce a low-pH mixture having a pH of about 4.5;
mildly agitating the low-pH mixture;
separating the low-pH mixture into a curd and a whey;
diluting the curd with water and mildly agitating to achieve a 6% dry solids mixture;
separating the 6% dry solids mixture into a second curd and a second whey;
diluting the second curd with water and mixing with sodium hydroxide to produce a 10% dry solids mixture having a pH of about 7.0;
pasteurizing the 10% dry solids mixture using a direct steam injection heater to produce a heated slurry; and
cooling the heated slurry with a flash cooling system using a vacuumed tank to produce a seed protein isolate; and
spray drying the seed protein isolate to produce a seed protein isolate powder product having a moisture content of about 5.5%.

22. The process of claim 20 wherein the soy protein concentrate comprises at least 67% protein and at most 8% acid hydrolysis fat.

23. The process of claim 14 wherein the soy protein concentrate comprises at least 65% protein and at most 10% acid hydrolysis fat.

24. The process of claim 8 wherein the soy protein isolate comprises at least 85% protein and at most 10% acid hydrolysis fat.

25. The process of claim 15 wherein pressing the seed material comprises feeding the seed material into a screw press and injecting carbon dioxide into the screw press.

26. The process of claim 15 wherein pressing the seed material comprises feeding the seed material into a screw press and injecting a non-carbon gas into the screw press.
